(19) United States
(12) Patent Application Publication
(10) Pub. No.: US 2025/0128070 A1
(43) Pub. Date: Apr. 24, 2025

(54) METHOD AND APPARATUS FOR USING STIMULATION AND SENSING DISTANCES IN NEUROSTIMULATION CONTROL

(71) Applicant: Boston Scientific Neuromodulation Corporation, Valencia, CA (US)

(72) Inventor: Michael A. Moffitt, Solon, OH (US)

(21) Appl. No.: 18/915,196

(22) Filed: Oct. 14, 2024

Related U.S. Application Data
(60) Provisional application No. 63/545,289, filed on Oct. 23, 2023.

(51) Int. Cl. A61N 1/36; A61N 1/05

(52) U.S. Cl. CPC A61N 1/36139; A61N 1/0551

(57) ABSTRACT

A neurostimulation system may include a stimulation output circuit to deliver neurostimulation pulses using a stimulation electrode positioned at a stimulation distance from a neural target, a sensing circuit to sense neural signals using a sensing electrode positioned at a sensing distance from the neural target, and a stimulation control circuit that may include a test controller and a test processor. The test controller may be configured to control performance of an electrode distance test including sensing a first neural signal of the neural signals using the sensing electrode while delivering the neurostimulation pulses using the stimulation electrode and sensing a second neural signal of the neural signals using the stimulation electrode while delivering the neurostimulation pulses using the sensing electrode. The test processor may be configured to analyze the stimulation and sensing distances using the first and second neural signals.

### CLAIM OF PRIORITY

This application claims the benefit of U.S. Provisional Application No. 63/545,289, filed on Oct. 23, 2023, which is hereby incorporated by reference in its entirety.

### CROSS REFERENCE TO RELATED APPLICATION

This application is related to commonly assigned U.S. patent application Ser. No. 18/380,106, published as 2024/0123234, entitled “METHOD AND APPARATUS FOR DETERMINING TRUE NEURAL ACTIVATION CHANGES”, filed on Oct. 13, 2023, which claims the benefit of priority under 35 U.S.C. § 119 (e) of U.S. Provisional Patent Application Ser. No. 63/416,123, entitled “METHOD AND APPARATUS FOR DETERMINING TRUE NEURAL ACTIVATION CHANGES”, filed on Oct. 14, 2022, which are incorporated by reference herein in their entireties.

### TECHNICAL FIELD

This document relates generally to neurostimulation and more particularly to a neurostimulation system can analyze distances from a stimulation electrode to a neural target and from a sensing electrode to the neural target in a patient and using an outcome of the analysis in controlling delivery of neurostimulation to the patient.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered to a patient in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. The stimulation parameters may be set and/or adjusted based on signals sensed from the patient and indicative of the patient's conditions and/or the patient's response to the delivery of the neurostimulation. For example, a closed-loop control system may sense the patient's neural responses to the delivery of the neurostimulation and adjust one or more stimulation parameters in response to a change in the sensed neural responses. The change in the sensed neural responses may attribute to a change in the patient's neural activity and/or a change in positions of stimulation and sensing electrodes relative to a neural target to which the neurostimulation is delivered and from which the neural responses are sensed.

### SUMMARY

An Example (e.g., “Example 1”) of a system for delivering neurostimulation to a neural target in a patient using a plurality of electrodes is provided. The system may include a stimulation output circuit, a sensing circuit, and a stimulation control circuit. The stimulation output circuit may be configured to deliver neurostimulation pulses to the neural target using a stimulation electrode selected from the plurality of electrodes. The stimulation electrode is positioned at a stimulation distance from the neural target. The sensing circuit may be configured to sense neural signals from the neural target using a sensing electrode selected from the plurality of electrodes. The sensing electrode is positioned at a sensing distance from the neural target. The neural signals include neural responses each evoked by the delivery of a pulse of the neurostimulation pulses. The stimulation control circuit may be configured to control the delivery of the neurostimulation pulses. The stimulation control circuit may include a test controller and a test processor. The test controller may be configured to control performance of an electrode distance test including sensing a first neural signal of the neural signals using the sensing electrode while delivering the neurostimulation pulses using the stimulation electrode and sensing a second neural signal of the neural signals using the stimulation electrode while delivering the neurostimulation pulses using the sensing electrode. The test processor may be configured to analyze the stimulation distance and the sensing distance using the first neural signal and the second neural signal.

In Example 2, the subject matter of Example 1 may optionally be configured such that the stimulation control circuit includes a control adjuster configured to adjust the control of the delivery of the neurostimulation pulses using an outcome of the analysis.

In Example 3, the subject matter of any one or any combination of Examples 1 and 2 may optionally be configured to further include an implantable neurostimulator including the stimulation output circuit, the sensing circuit, and the stimulation control circuit.

In Example 4, the subject matter of Example 3 may optionally be configured to further include an implantable lead including the plurality of electrodes.

In Example 5, the subject matter of any one or any combination of Examples 1 to 4 may optionally be configured such that the test controller is configured to control the delivery of the neurostimulation pulses during the electrode distance test using stimulation parameters determined to minimize perception of stimulation by the patient.

In Example 6, the subject matter of any one or any combination of Examples 1 to 5 may optionally be configured such that the test processor is configured to determine a first magnitude being a magnitude of the neural responses in the first neural signal, to determine a second magnitude being a magnitude of the neural responses in the second neural signal, and to analyze the stimulation distance and the sensing distance using the first magnitude and the second magnitude.

In Example 7, the subject matter of Example 6 may optionally be configured such that the test processor is configured to determine, as an outcome of the analysis, whether the stimulation distance and the sensing distance are approximately equal.

In Example 8, the subject matter of Example 7 may optionally be configured such that the test processor is configured to determine, as an outcome of the analysis if the stimulation distance and the sensing distance are not approximately equal, at least one of: a degree of difference between the stimulation distance and the sensing distance, or which of the stimulation and sensing electrodes is closer to the neural target if the stimulation distance and the sensing distance are not approximately equal.

In Example 9, the subject matter of any one or any combination of Examples 6 to 8 may optionally be configured such that the test processor includes a detection module, a measurement module, and an analysis module. The detection module is configured to detect the neural responses including morphological features of the neural responses from each of the first neural signal and the second neural signal. The measurement module is configured to determine a first neural response parameter using the morphological features detected from the first neural signal and a second neural response parameter using the morphological features detected from the second neural signal. The analysis module is configured to analyze the stimulation distance and the sensing distance using the first neural response parameter and the second neural response parameter.

In Example 10, the subject matter of Example 9 may optionally be configured such that the neural signals include evoked compound action potentials (ECAPs) each evoked by the delivery of a pulse of the neurostimulation pulses, the detection module is configured to detect ECAPs including ECAP features each being a morphological feature of the ECAPs from each of the first neural signal and the second neural signal, and the measurement module is configured to determine the first neural response parameter using the ECAP features detected from the first neural signal and the second neural response parameter using the ECAP features detected from the second neural signal.

In Example 11, the subject matter of any one or any combination of Examples 1 to 10 may optionally be configured such that the stimulation control circuit further includes a test initiator configured to initiate the electrode distance test in response to an initiation command.

In Example 12, the subject matter of Example 11 may optionally be configured such that the test initiator is configured to detect a change in the sensed neural signals and to initiate the electrode distance test in response to the change exceeding a specified threshold.

In Example 13, the subject matter of any one or any combination of Examples 11 and 12 may optionally be configured to further include an accelerometer configured to sense a movement or posture of the patient and to produce an accelerometer signal indicative of the movement or posture, and such that the test initiator is configured to detect a change in the accelerometer signal and to initiate the electrode distance test in response to the change exceeding a specified threshold.

In Example 14, the subject matter of any one or any combination of Examples 11 to 13 may optionally be configured such that the test initiator is configured to monitor adjustments of one or more parameters of the set of stimulation parameters by the patient and to initiate the electrode distance test in response to one of the one or more parameters being adjusted by the patient.

In Example 15, the subject matter of any one or any combination of Examples 1 to 14 may optionally be configured such that the stimulation control circuit further includes a notification generator to produce a notification using an outcome of the analysis.

An example (e.g., “Example 16”) of a method for delivering neurostimulation to a neural target in a patient is also provided. The method may include: delivering neurostimulation pulses to the neural target using a stimulation electrode of a plurality of electrodes, sensing neural signals from the neural target using a sensing electrode of the plurality of electrodes, performing an electrode distance test including sensing a first neural signal of the neural signals using the sensing electrode while delivering the neurostimulation pulses using the stimulation electrode and sensing a second neural signal of the neural signals using the stimulation electrode while delivering the neurostimulation pulses using the sensing electrode, and analyzing the stimulation distance and the sensing distance using the first neural signal and the second neural signal. The stimulation electrode is positioned at a stimulation distance from the neural target. The sensing electrode is positioned at a sensing distance from the neural target. The neural signals include neural responses each evoked by the delivery of a pulse of the neurostimulation pulses.

In Example 17, the subject matter of Example 16 may optionally further include adjusting control of the delivery of the neurostimulation pulses using an outcome of the analysis.

In Example 18, the subject matter of analyzing the stimulation distance and the sensing distance using the first neural signal and the second neural signal as found in any one or any combination of Examples 16 and 17 may optionally include: determining a first magnitude being a magnitude of the neural responses in the first neural signal, determining a second magnitude being a magnitude of the neural responses in the second neural signal, and analyzing the stimulation distance and the sensing distance using the first magnitude and the second magnitude.

In Example 19, the subject matter of analyzing the stimulation distance and the sensing distance as found in Example 18 may optionally include determining whether the stimulation distance and the sensing distance are approximately equal.

In Example 20, the subject matter of analyzing the stimulation distance and the sensing distance as found in Example 19 may optionally include determining, if the stimulation distance and the sensing distance are not approximately equal, at least one of: a degree of difference between the stimulation distance and the sensing distance, or which of the stimulation and sensing electrodes is closer to the neural target.

In Example 21, the subject matter of determining the first magnitude and determining the second magnitude as found in any one or any combination of Examples 18 to 20 may optionally include: detecting the neural responses including morphological features of the neural responses from each of the first neural signal and the second neural signal, determining a first neural response parameter as the first magnitude using the morphological features detected from the first neural signal, and determining a second neural response parameter as the second magnitude using the morphological features detected from the second neural signal.

In Example 22, the subject matter of sensing the neural signals as found in Example 21 may optionally include sensing neural signals including evoked compound action potentials (ECAPs) each evoked by the delivery of a pulse of the neurostimulation pulses, the subject matter of detecting the neural responses as found in Example 21 may optionally include detecting ECAP features each being a morphological feature of the ECAPs, the subject matter of determining the first neural response parameter as found in Example 21 may optionally include determining the first neural response parameter using the ECAP features detected from the first neural signal, and the subject matter of determining the second neural response parameter as found in Example 21 may optionally include determining the second neural response parameter using the ECAP features detected from the second neural signal.

In Example 23, the subject matter of any one or any combination of Examples 16 to 22 may optionally further include initiating the performance of the electrode distance test in response to at least one of a user command for performing the electrode distance test, detection of a change in the sensed neural signals exceeding a sensing threshold, detection of a change in an accelerometer signal indicative of movement or posture of the patient exceeding a movement threshold, or an adjustment of one or more specified parameters of the set of stimulation parameters being made by the patient.

In Example 24, the subject matter of any one or any combination of Examples 16 to 23 may optionally further include producing a notification using an outcome of the analysis.

An example (e.g., “Example 25”) of a non-transitory computer-readable storage medium including instructions is also provided. The instructions, which when executed by a system, cause the system to perform a method for delivering neurostimulation to a neural target in a patient. The method may include: delivering neurostimulation pulses to the neural target using a stimulation electrode of a plurality of electrodes, sensing neural signals from the neural target using a sensing electrode of the plurality of electrodes, performing an electrode distance test including sensing a first neural signal of the neural signals using the sensing electrode while delivering the neurostimulation pulses using the stimulation electrode and sensing a second neural signal of the neural signals using the stimulation electrode while delivering the neurostimulation pulses using the sensing electrode, and analyzing the stimulation distance and the sensing distance using the first neural signal and the second neural signal. The stimulation electrode is positioned at a stimulation distance from the neural target. The sensing electrode is positioned at a sensing distance from the neural target. The neural signals include neural responses each evoked by the delivery of a pulse of the neurostimulation pulses.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an example of a lead including an electrode array placed over the spinal cord of a patient.

FIGS. 10A-10C illustrate various examples of displacement of the electrode array of FIG. 9 relative to the spinal cord, with FIG. 10A showing examples of equal stimulation and sensing distances, FIG. 10B showing examples of stimulation distance greater than sensing distance, and FIG. 10C showing examples of stimulation distance shorter than sensing distance.

FIGS. 11A-11C illustrate various examples of neural responses to neurostimulation at various displacements of the electrode array of FIG. 9 relative to the spinal cord, with FIG. 11A showing examples with a fixed stimulation distance and a set of different sensing distances, FIG. 11B showing examples with another fixed stimulation distance and the set of different sensing distances, and FIG. 11C showing examples of yet another fixed stimulation distance and the set of different sensing distances.

FIG. 12 illustrates examples of evoked compound action potential (ECAP) features of a neural signal and examples of neural response parameters produced using the ECAP features.

FIG. 13 illustrates an embodiment of a system for delivering neurostimulation and sensing a neural signal that can perform an electrode distance test, such as may be implemented in the neurostimulation system of FIG. 1.

FIG. 14 illustrates an embodiment of a stimulation control circuit of a neurostimulation system, such as the system of FIG. 13.

FIG. 15 illustrates an embodiment of a test processor of a stimulation control circuit, such as the stimulation control circuit of FIG. 14.

FIG. 16 illustrates another embodiment of the stimulation control circuit of FIG. 14.

FIG. 17 illustrates an embodiment of a method for delivering neurostimulation, including performing an electrode distance test and controlling the delivery of the neurostimulation using a result of the electrode distance test.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a neurostimulation system that can deliver neurostimulation through a stimulation electrode to a neural target in a patient, sense a neural signal through a sensing electrode from the neural target, analyze a distance between the stimulation electrode relative to a distance between the sensing electrode using the sensed neural signal, and control delivery of neurostimulation to the patient using an outcome of the analysis. In various embodiments, the neuromodulation system can include an implantable device configured to deliver neurostimulation (also referred to as neuromodulation) therapies, such as spinal cord stimulation (SCS), deep brain stimulation (DBS), peripheral nerve stimulation (PNS), and vagus nerve stimulation (VNS), and one or more external devices configured to program or adjust the implantable device for its operations and monitor the performance of the implantable device. In this document, unless noted otherwise, a “patient” includes a person receiving treatment delivered from, and/or monitored using, a neurostimulation system according to the present subject matter. A “user” includes a physician, other caregiver who examines and/or treats the patient using the neurostimulation system, or other person who participates in the examination and/or treatment of the patient using the neurostimulation system (e.g., a technically trained representative, a field clinical engineer, a clinical researcher, or a field specialist from the manufacturer of the neurostimulation system).

In SCS, one or more leads may be used to deliver neurostimulation and sense a neural signal with sensing and stimulation electrodes epidurally placed on the spinal cord of a patient. The stimulation and sensing locations can be separated by around 15 mm or more to reduce stimulus artifacts in sensing of neural responses such as the evoked compound action potentials (ECAPs). In various examples of an existing SCS system that controls delivery of neurostimulation using stimulation parameters that are adjusted according to an ECAP-based closed-loop control algorithm, it is assumed that the distance between the spinal cord and the stimulation electrode (herein referred to as “stimulation distance”, or “dₛₜᵢₘ”) and the distance between the spinal cord and the sensing electrode (herein referred to as “sensing distance”, or “dₛₑₙₛₑ”) are equal (i.e., dₛₜᵢₘ=dₛₑₙₛₑ). However, body movements of the patient causes the spinal cord to move within the cerebrospinal fluid (CSF) and causes shifts in the epidurally placed sensing and stimulation electrodes. Using a computational ECAP model of the spinal cord, responses of dorsal column fibers to various stimulation parameters and for various combinations of the stimulation distance and the sensing distance (including various instances for each of dₛₜᵢₘ=dₛₑₙₛₑ, dₛₜᵢₘ>dₛₑₙₛₑ, and dₛₜᵢₘ<dₛₑₙₛₑ) were determined by simulations. The result demonstrated that the assumption of equal stimulation distance and sensing distance could lead to erroneous adjustments of stimulation parameters because an adjustment could be made in response to a change in the sensed neural signal that is caused by a body movement of the patient rather than a change in the patient's neural activation.

The present subject matter allows for analysis of a stimulation distance relative to a sensing distance and adjustment of stimulation parameters for controlling the delivery of the neurostimulation using an outcome of the analysis. The stimulation distance is the distance between a stimulation electrode and the neural target in the patient. The sensing distance is the distance between a stimulation electrode and the neural target. A first neural signal is sensed using the sensing electrode while the neurostimulation is delivered using the stimulation electrode. A second neural signal is sensed using the stimulation electrode while the neurostimulation is delivered using the sensing electrode. The stimulation distance and the sensing distance can then be analyzed using the first neural signal and the second neural signal. Outcome of the analysis can be used to account for the electrode-to-neural target distances in control of neurostimulation, such as controlling stimulation parameters and/or algorithms for controlling the stimulation parameters. Examples of the stimulation parameters that can be adjusted include stimulation current (pulse amplitude), frequency (also referred to as rate), charge per second, charge per phase, pulse width, burst cycling times, and interphase interval (time between stimulation and recharging phases). An example of the algorithms for controlling the stimulation parameters includes a feedback control algorithm that adjusts the stimulation parameters using a sensed neural signal. In various embodiments, any one or any combination of such stimulation parameters and control algorithms (e.g., parameters used in the algorithms) can be adjusted for controlling neurostimulation according to the present subject matter.

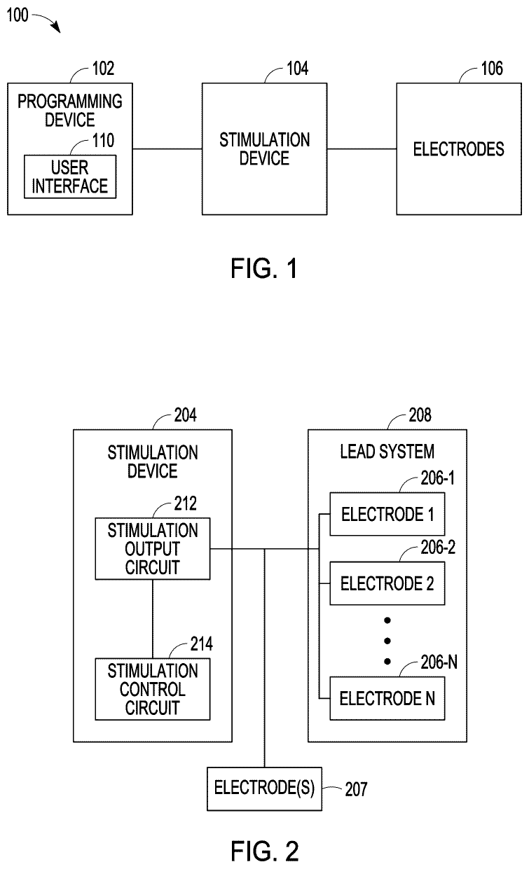

FIG. 1 illustrates an embodiment of a neurostimulation system 100. System 100 includes electrodes (also referred to as contacts) 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for spinal cord stimulation (SCS) applications. Such SCS configuration includes various features that may simplify the task of the user in programming stimulation device 104 for delivering SCS to the patient, such as the features discussed in this document.

FIG. 2 illustrates an embodiment of a stimulation device 204 and a lead system 208, such as may be implemented in neurostimulation system 100. Stimulation device 204 represents an example of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 (also referred to as an electrode array in this document) distributed in the one or more leads. The plurality of electrodes 206 includes electrode 206-1, electrode 206-2, . . . electrode 206-N, each being a single electrically conductive contact providing for an electrical interface between stimulation output circuit 212 and tissue of the patient (and therefore also referred to as a contact), where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

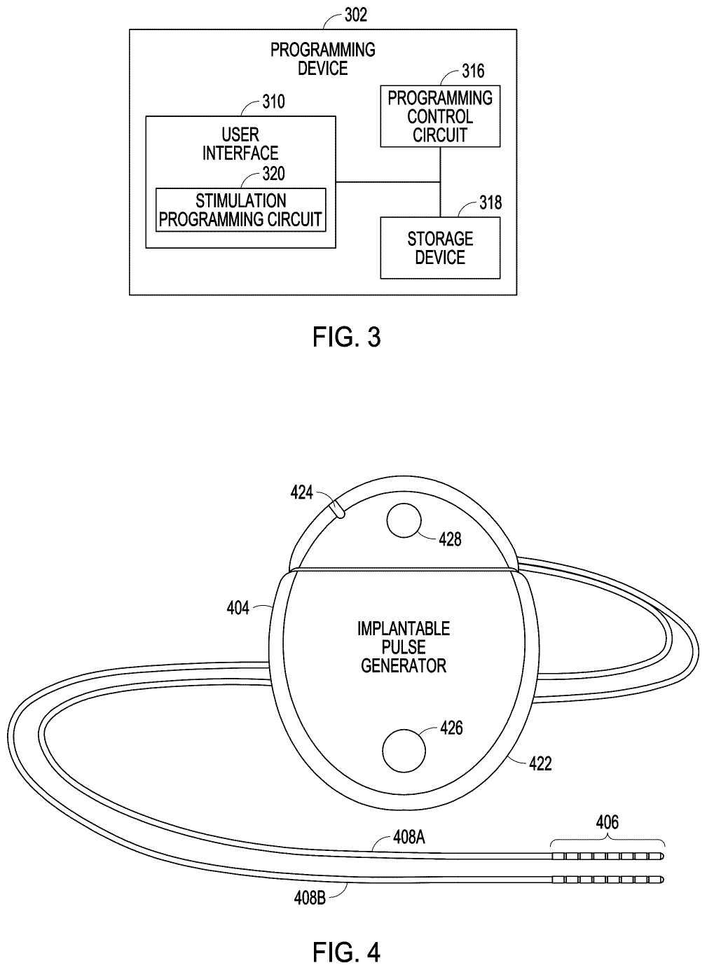

FIG. 3 illustrates an embodiment of a programming device 302, such as may be implemented in neurostimulation system 100. Programming device 302 represents an example of programming device 102 and includes a storage device 318, a programming control circuit 316, and a user interface 310. Programming control circuit 316 generates the plurality of stimulation parameters that controls the delivery of the neurostimulation pulses according to a specified neurostimulation program that can define, for example, stimulation waveform and electrode configuration. User interface 310 represents an example of user interface 110 and includes a stimulation programming circuit 320. Storage device 318 stores information used by programming control circuit 316 and stimulation programming circuit 320, such as information about a stimulation device that relates the neurostimulation program to the plurality of stimulation parameters. In various embodiments, stimulation programming circuit 320 can be configured to support one or more functions allowing for programming of stimulation devices, such as stimulation device 104 including its various embodiments as discussed in this document, to control delivery of neurostimulation according to the present subject matter (e.g., analyzing stimulation and sensing distances and using an outcome of the analysis to adjust the control of the delivery of neurostimulation).

In various embodiments, user interface 310 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “neurostimulation program” can include the pattern of neurostimulation pulses including the one or more stimulation fields, or at least various aspects or parameters of the pattern of neurostimulation pulses including the one or more stimulation fields. In various embodiments, user interface 310 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

In various embodiments, circuits of neurostimulation system 100, including its various embodiments discussed in this document, may be implemented using a combination of hardware and software. For example, the circuit of user interface 110, stimulation control circuit 214, programming control circuit 316, and stimulation programming circuit 320, including their various embodiments discussed in this document, can be implemented using an application-specific circuit constructed to perform one or more particular functions and/or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrodes 406 for contacting a tissue site targeted for electrical neurostimulation. As illustrated in FIG. 4, leads 408A and 408B each include 8 electrodes 406 at the distal end. The number and arrangement of leads 408A and 408B and electrodes 406 as shown in FIG. 4 are only an example, and other numbers and arrangements are possible. In various embodiments, the electrodes are ring electrodes. In various embodiments applying DBS or SCS, the implantable leads and electrodes may be configured by shape and size to provide electrical neurostimulation energy to a neuronal target included in the patient's brain or configured to provide electrical neurostimulation energy to target nerve cells in the patient's spinal cord.

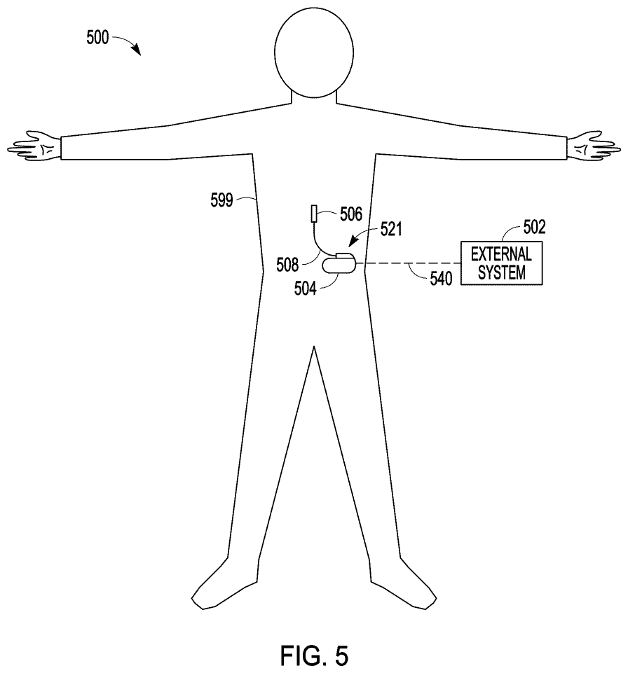

FIG. 5 illustrates an implantable neurostimulation system 500 and portions of an environment in which system 500 may be used. System 500 includes an implantable system 521, an external system 502, and a telemetry link 540 providing for wireless communication between implantable system 521 and external system 502. Implantable system 521 is illustrated in FIG. 5 as being implanted in the patient's body 599.

Implantable system 521 includes an implantable stimulator (also referred to as an implantable pulse generator, or IPG) 504, a lead system 508, and electrodes 506, which represent an example of stimulation device 204, lead system 208, and electrodes 206, respectively. External system 502 represents an example of programming device 302. In various embodiments, external system 502 includes one or more external (non-implantable) devices each allowing the user and/or the patient to communicate with implantable system 521. In some embodiments, external 502 includes a programming device intended for the user to initialize and adjust settings for implantable stimulator 504 and a remote control device intended for use by the patient. For example, the remote control device may allow the patient to turn implantable stimulator 504 on and off and/or adjust certain patient-programmable parameters of the plurality of stimulation parameters.

The sizes and shapes of the elements of implantable system 521 and their location in body 599 are illustrated by way of example and not by way of restriction. An implantable system is discussed as a specific application of the programming according to various embodiments of the present subject matter. In various embodiments, the present subject matter may be applied in programming any type of stimulation device that uses electrical pulses as stimuli, regarding less of stimulation targets in the patient's body and whether the stimulation device is implantable.

Returning to FIG. 4, the IPG 404 can include a hermetically-sealed IPG case 422 to house the electronic circuitry of IPG 404. IPG 404 can include an electrode 426 formed on IPG case 422. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode. Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

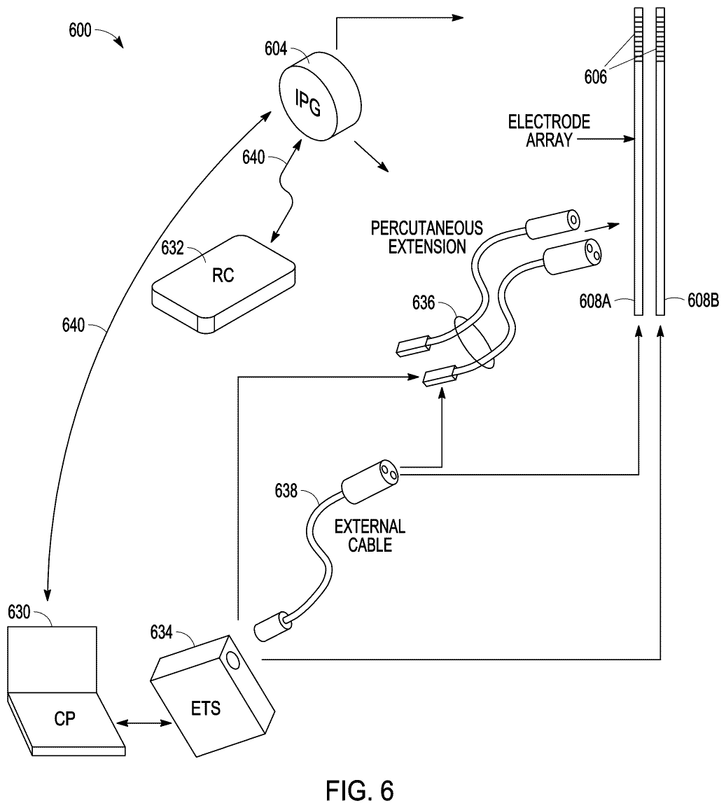

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (ETS, also referred to as external trial modulator, ETM) 634. JPG 604 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an example of system 100, with JPG 604 representing an embodiment of stimulation device 104, electrodes 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604.

RC 632 also communicates with IPG 604 using a wireless link 640. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630. CP 630 may communicate with the RC 632 using a wired or wireless communications link. In some embodiments, CP 630 can program RC 632 when remotely located from RC 632. In various embodiments, RC632 can be a dedicated device or a general-purpose device configured to perform the functions of RC 632, such as a smartphone, a tablet computer, or other smart/mobile device.

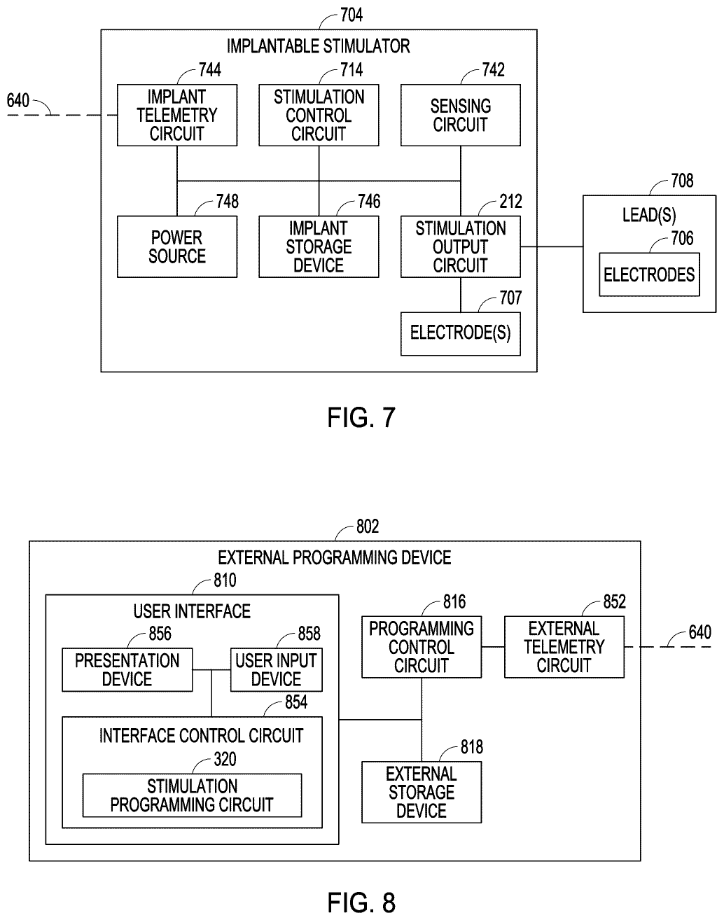

FIG. 7 illustrates an embodiment of implantable stimulator 704 and one or more leads 708 of an implantable neurostimulation system, such as implantable system 600. Implantable stimulator 704 represents an example of stimulation device 104 or 204 and may be implemented, for example, as IPG 604. Lead(s) 708 represents an example of lead system 208 and may be implemented, for example, as implantable leads 608A and 608B. Lead(s) 708 includes electrodes 706, which represents an example of electrodes 106 or 206 and may be implemented as electrodes 606.

Implantable stimulator 704 may include a sensing circuit 742 that provides the stimulator with a sensing capability, stimulation output circuit 212, a stimulation control circuit 714, an implant storage device 746, an implant telemetry circuit 744, a power source 748, and one or more electrodes 707. Sensing circuit 742 can one or more physiological signals for purposes of patient monitoring and/or feedback control of the neurostimulation. In various embodiments, sensing circuit 742 can sense one or more electrospinogram (ESG) signals using electrodes placed over or under the dura of the spinal cord, such as electrodes 706 (which can include epidural and/or intradural electrodes). In addition to one or more ESG signals, examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707 and delivers each of the neurostimulation pulses through a set of electrodes selected from electrodes 706 and electrode(s) 707. Stimulation control circuit 714 represents an example of stimulation control circuit 214 and controls the delivery of the neurostimulation pulses using the plurality of stimulation parameters specifying the pattern of neurostimulation pulses. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 can store one or more neurostimulation programs and values of the plurality of stimulation parameters for each of the one or more neurostimulation programs. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 (e.g., by installing a new application in a smart device such as a smartphone) and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640 and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing circuit 742.

In various embodiments, sensing circuit 742 (if included), stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an example of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). This can allow for patient mobility during programming and assessment when needed. For example, wireless communication link 640 can include at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a DBS or SCS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through which a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time to shape the stimulation like a piece of clay (pushing/pulling one spot at a time).

Programming control circuit 816 represents an example of programming control circuit 316 and generates the plurality of stimulation parameters, which is to be transmitted to implantable stimulator 704, based on a specified neurostimulation program (e.g., the pattern of neurostimulation pulses as represented by one or more stimulation waveforms and one or more stimulation fields, or at least certain aspects of the pattern). The neurostimulation program may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an example of user interface 310 and allows the user to define the pattern of neurostimulation pulses and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes stimulation programming circuit 320.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. Programming control circuit 816 dynamically updates values of the plurality of stimulation parameters in response to changes in the set of one or more stimulation waveforms and transmits the plurality of stimulation parameters with the updated values to implantable stimulator 704.

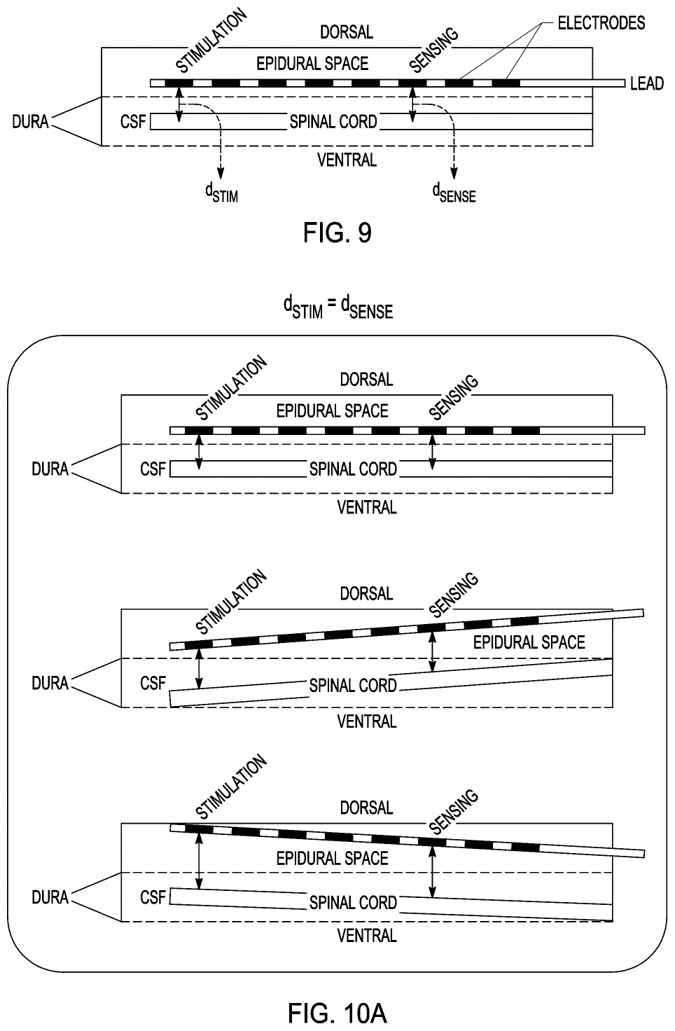

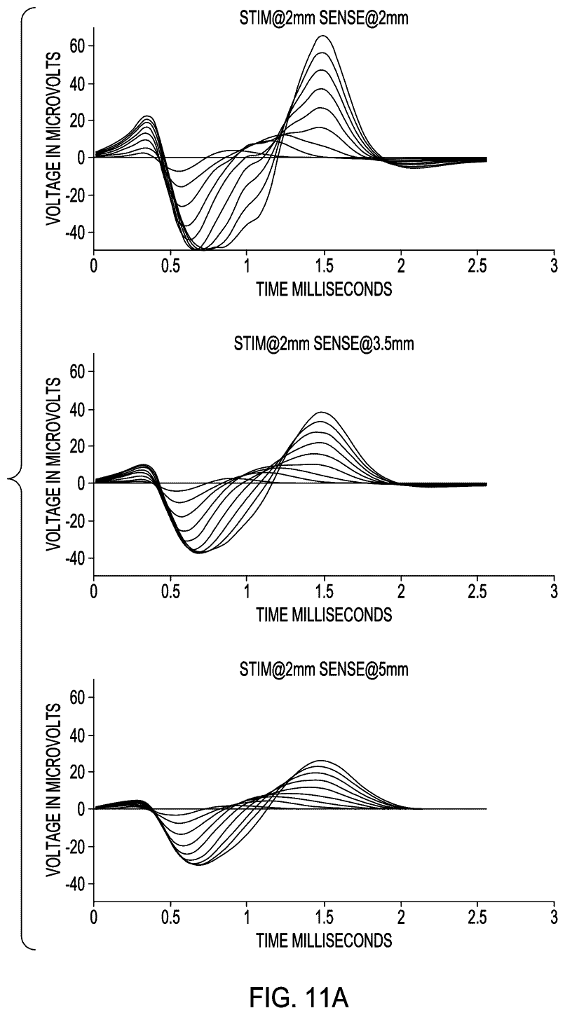

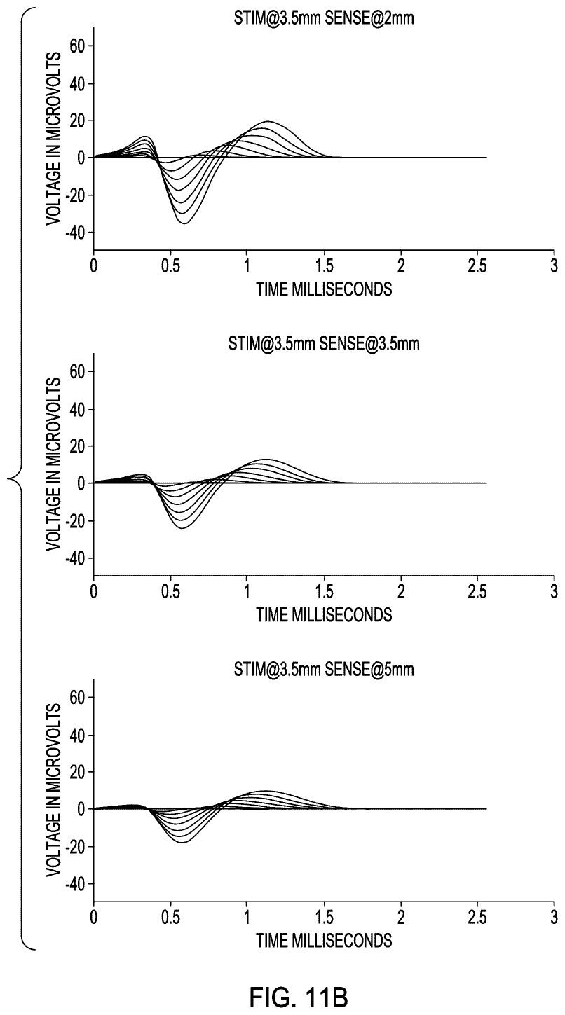

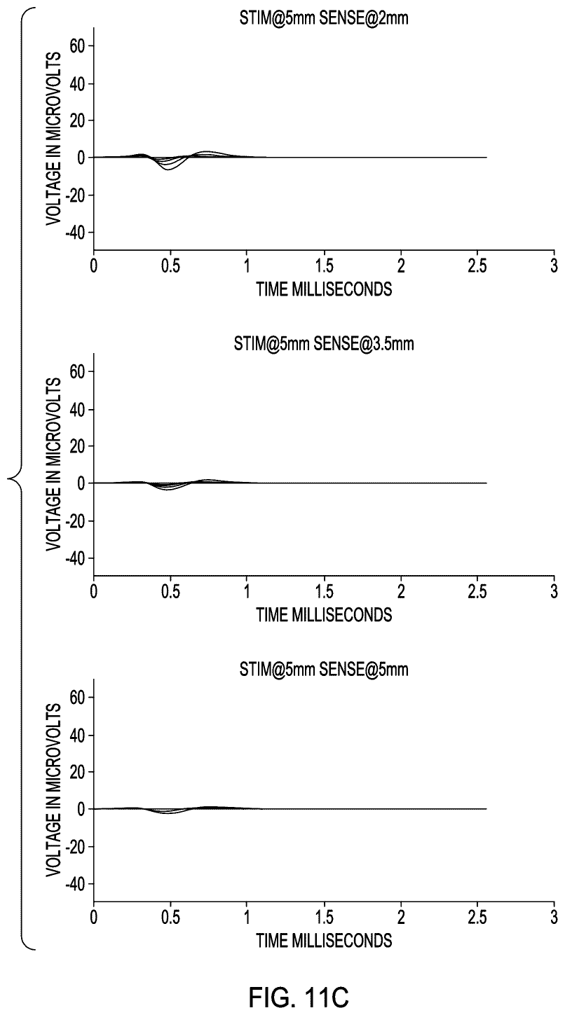

FIGS. 9-16 illustrate examples of effects of the patient's body movements on neural responses during SCS. In various embodiments, a neural signal is sensed and used in closed-loop control of delivery of neurostimulation in which the patient's neural activation changes should be responded by adjusting one or more stimulation parameters. Changes in the sensed neural signal are used to indicate the patient's neural activation changes, but factors other than the neural activation changes can also affect the sensed neural signal. Such factors need to be excluded when determining whether a change in the sensed neural signal should trigger an adjustment of the stimulation parameter(s). A computational ECAP model of the spinal cord was used for studying the effects of the patient's body movements on neural responses using simulations. Examples of results produced by the simulation are shown in FIGS. 11 and 13-16.

FIG. 9 illustrates an example of a lead including an array of electrodes placed over the spinal cord of a patient for delivering SCS to the patient. The spinal cord is in cerebrospinal fluid (CSF) surrounded by the dura mater (dura). The lead as shown in FIG. 9 has eight electrodes (also referred to as contacts) placed in the epidural space over the spinal cord. These electrodes can each be used as a stimulation electrode (though which neurostimulation is delivered to the patient) and/or a sensing electrode (through which a neural signal is sensed from the patient). FIG. 9 shows, as an example, that the first electrode from the left is used as a stimulation electrode, and the sixth electrode from the left is used as a sensing electrode. The distance between the stimulation electrode and the spinal cord is referred to as the stimulation distance (dₛₜᵢₘ). The distance between the sensing electrode and the spinal cord is referred to as the sensing distance (dₛₑₙₛₑ). FIG. 9 shows an ideal case in which the stimulation distance and the sensing distance are equal (dₛₜᵢₘ=dₛₑₙₛₑ) in the dorsal-ventral (DV) direction.

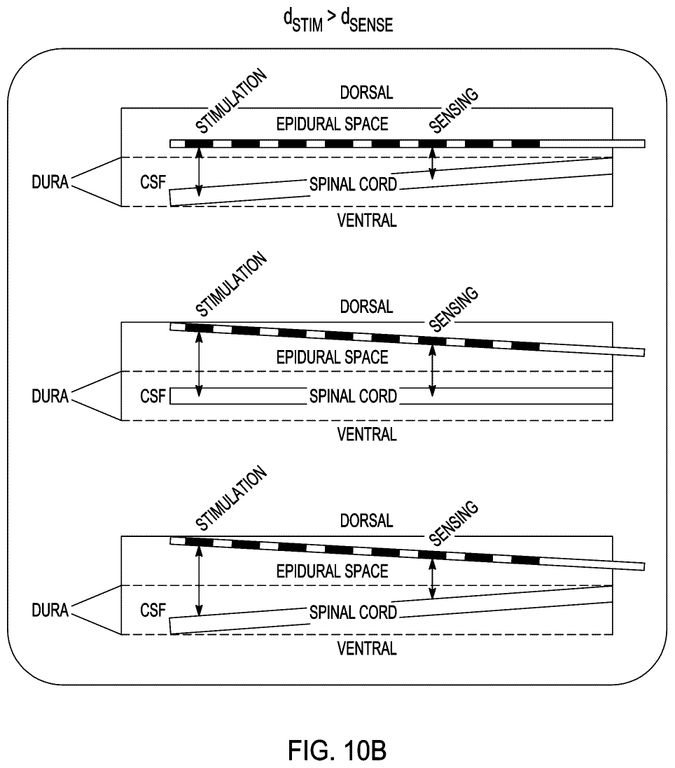

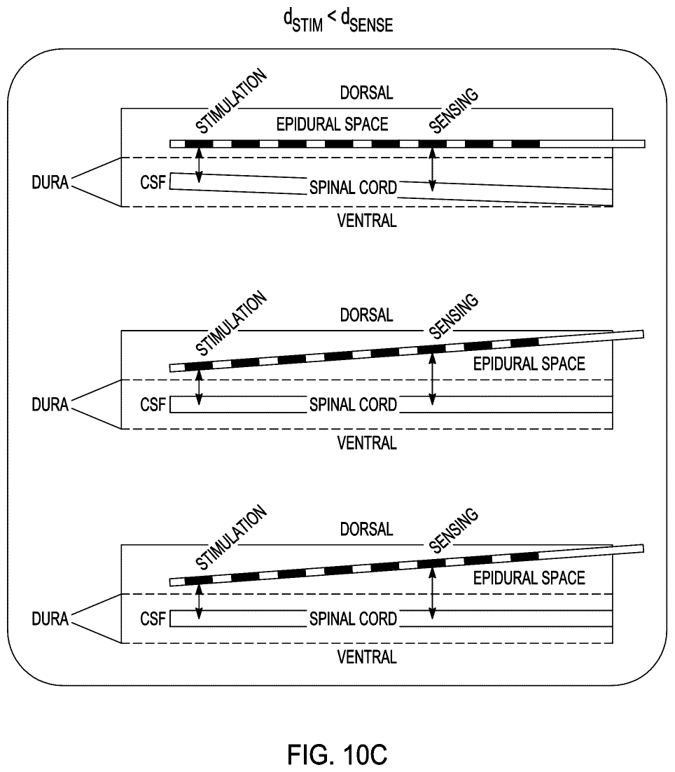

FIGS. 10A-10C illustrate various examples of displacement of the electrode array of FIG. 9 relative to the spinal cord. These examples illustrate various combinations of the stimulation distance and the sensing distance that may result from the patient's body movements. Such movements can cause the spinal cord to move within the CSF and/or the epidurally placed lead (including the sensing and stimulation electrodes to shift locations. The various combinations as illustrated in FIG. 10A includes cases of spinal cord movement and/or lead shifting resulting in substantially equal stimulation and sensing distances (dₛₜᵢₘ=dₛₑₙₛₑ). FIG. 10B includes cases of spinal cord movement and/or lead shifting resulting in the stimulation distance substantially greater than the sensing distance (dₛₜᵢₘ>dₛₑₙₛₑ). FIG. 10C include cases of spinal cord movement and/or lead shifting resulting in the stimulation distance substantially shorter than the sensing distance (dₛₜᵢₘ<dₛₑₙₛₑ). All these combinations can be experiences in the practice of SCS.

FIGS. 11A-11C illustrate various examples of neural responses to neurostimulation at various displacements of the electrode array of FIG. 9 relative to the spinal cord with FIG. 11A showing examples with a fixed stimulation distance and a set of different sensing distances, FIG. 11B showing examples with another fixed stimulation distance and the set of different sensing distances, and FIG. 11C showing examples of yet another fixed stimulation distance and the set of different sensing distances. These examples show sensed neural responses including ECAPs with different values of the stimulation distance and the sensing distance (“Stim@Xmm Sense@Ymm” means dₛₜᵢₘ=X mm and dₛₑₙₛₑ=Y mm). The significant morphological differences in these sensed neural responses suggest that morphological features of the neural response (including ECAP features) in the sensed neural signal can be used to indicate changes in the sensed signal that attribute to changes in the stimulation distance and/or the sensing distance (rather than changes attributing to neural activation change).

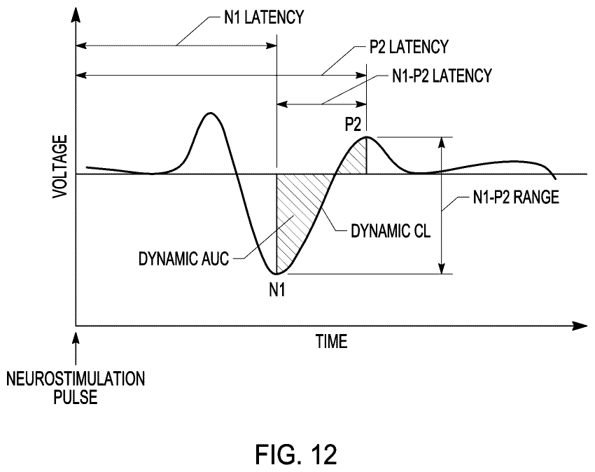

FIG. 12 illustrates examples of ECAP features of a neural signal and examples of neural response parameters produced using the ECAP features. The signal shown in FIG. 12 is an example of a neural response that is evoked by a neurostimulation pulse and includes an ECAP indicative of dorsal column response (e.g., as seen on an electrospinographic signal). Morphological features of the ECAP (herein referred to as “ECAP features”) that can be used in the present subject matter include, but are not limited to:
N1: the first negative peak in an evoked response that is correlated to the response of faster fibers such as Aβ fibers and myelinated fibers; and
P2: the second positive peak in the evoked response that is correlated with response of slower fibers.
   Various neural response parameters can be derived from characteristics of the evoked responses as seen on the neural signal. In various embodiments, one or more neural response parameters can be measured from the neural signal using the ECAP features. Examples of the one or more neural response parameters generated by detecting and measuring ECAP features for analyzing neural activation and/or controlling delivery of neurostimulation include:
N1 amplitude: amplitude of N1 (measured from a base line, e.g., 0 V);
P1 amplitude: amplitude of P1 (measured from the base line);
N1-P2 range: N1 to P2 amplitude (the difference between amplitudes of N1 and P2);
N1-P2 latency: time interval between N1 and P2;
N1 latency: time interval between start of recording frame (e.g., the neurostimulation pulse) and N1;
P2 latency: time interval between start of recording frame (e.g., the neurostimulation pulse) and P2;
Dynamic curve length (CL): curve length measured from the sensed neural signal between N1 and P2; and
Dynamic area under the curve (AUC): the area between the sensed neural signal and a baseline, measured between N1 and P2.
   In addition to or in place of the time-domain parameters above, the one or more neural response parameters can also include, for example:
Spectral power parameters (e.g. peak amplitude of each ECAP feature and/or total AUC of ECAP power band from 500 Hz to 3000 Hz);
Parseval power parameters (integral of the spectrum squared over a relevant power band); and
Other metrics of energy or power.
   In various embodiments, one or more of these neural response parameters can be used in the analysis distinguishing changes in the sensed neural signal caused by the patient's body movements from true neural activation changes. While the ECAP features and neural response parameters illustrated in FIG. 12 are discussed as examples, other ECAPs and neural response parameters can be suitable and used in the present subject matter as determined by those skilled in the art.

It can be seen from FIGS. 11A-11C that the stimulation and sensing distances for a pair of stimulation and sensing electrodes affect magnitude of a neural signal sensed using the sensing electrode, with the neural signal including neural responses to neural stimulation delivered using the stimulation electrode. For example, the bottom graph of FIG. 11A shows a segment of a neural signal sensed at a sensing distance of 5 mm that includes a neural response evoked by a neurostimulation pulse delivered at a stimulation distance of 2 mm, and the top graph of FIG. 11C shows a segment of the neural signal sensed at a sensing distance of 2 mm that includes a neural response evoked by a neurostimulation pulse delivered at a stimulation distance of 5 mm, while the stimulation and sensing parameters are otherwise the same for both graphs. The substantial difference in the magnitude of the neural responses between these two cases show the effect of the stimulation and sensing distances can have in a sensed neural signal. A change in the magnitude (e.g., as measured by the amplitude, shape, and/or other related features or parameters such as those discussed above) of the neural responses can result from a change in the stimulation and sensing distances, rather than a change in the patient's neural activation. This indicates that, for example, when controlling delivery of the neural stimulation using the sensed neural signal as an input, it can be desirable, if not necessary, to account for the stimulation and sensing distances. The change in the stimulation and sensing distances can result from the patient's posture change and/or other physical reasons that occur regularly and frequently, so such change may need to be monitored, and its extent analyzed, regularly and frequently to ensure efficacy and safety of the neurostimulation. The magnitude of the neural responses can be measured, for example, using the neural response parameters measured from ECAP features, as discussed above with reference to FIG. 12, when the neural signal includes ECAPs, such as in the case of SCS with ESG sensed.

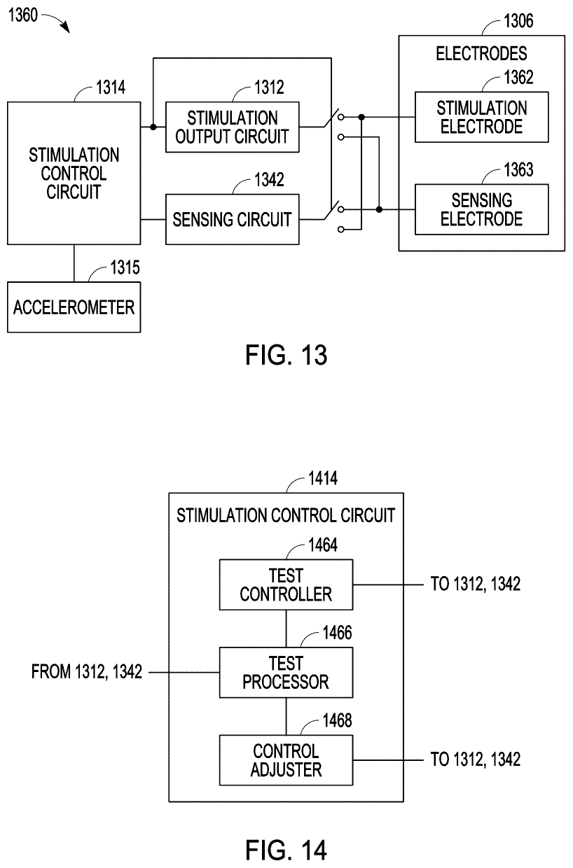

FIG. 13 illustrates an embodiment of a system 1360 for delivering neurostimulation to a neural target in the patient and sensing a neural signal from the neural target. System 1360 can perform an electrode distance test for analyzing distances between electrodes and the neural target. System 1360 includes a plurality of electrodes 1306, a stimulation output circuit 1312, a sensing circuit 1342, a stimulation control circuit 1314, and optionally an accelerometer 1315. Electrodes 1306 can include electrodes on one or more leads. As an example, FIGS. 9 and 10 show a lead including eight electrodes, with one used as a stimulation electrode and another used as a sensing electrode. In various embodiments, each electrode of electrodes 1306 can be selected for use as a stimulation electrode or a sensing electrode (e.g., as a stimulation period for a time period and a sensing period for a different time period). Stimulation output circuit 1312 can represent an example of stimulation output circuit 212 and can deliver neurostimulation pulses to the neural target using a stimulation electrode 1362 selected electrodes 1306. Stimulation electrode 1362 is positioned in the patient at a stimulation distance from the neural target. Sensing circuit 1342 can represent an example of sensing circuit 742 and can sense neural signals from the neural target using a sensing electrode 1363 selected from electrodes 1306. Sensing electrode 1363 is positioned at a sensing distance from the neural target. The neural signals include neural responses each evoked by the delivery of a pulse of the neurostimulation pulses from stimulation output circuit 1312. In one embodiment, the neural target is the patient's spinal cord. The neural signals include ESG signals including ECAPs each evoked by a pulse of the neurostimulation pulses. Stimulation control circuit 1314 can control the delivery of the neurostimulation pulses from stimulation output circuit 1312 and sensing of the neural signals using sensing circuit 1342. System 1360 can optionally include an accelerometer to sense a movement or posture of the patient and produce an accelerometer signal indicative of the movement or posture.

System 1360 can be implemented in neurostimulation systems such as systems 100, 500, and 600. In various embodiments, system 1360 can be implemented in an implantable medical device, such as IPG 404, IPG or implantable stimulator 504, IPG 604, or implantable stimulator 704, which can be communicatively coupled to and programmed by an external programming device, such as external system 502, CP 630, RC 632, or external programming device 802, as discussed in this document. For example, when system 1360 is implemented in implantable stimulator 704 that can be communicatively coupled to external programming device 802, lead(s) 708 can include electrodes 1306, stimulation output circuit 212 can be configured to include stimulation output circuit 1312, sensing circuit 742 can be configured to include sensing circuit 1342, and stimulation control circuit 714 can be configured to include stimulation control circuit 1314. Stimulation programming circuit 320 can be configured to support programming of implantable stimulator 704 for the operations of system 1360. In various embodiments, system 1360 can be implemented in a single implantable or non-implantable neurostimulator with parameters needed for its operation programmable using a programming device. In various other embodiments, system 1360 can be distributed in both the implantable or non-implantable neurostimulator and the programming device.

Stimulation control circuit 1314 can perform the electrode distance test for analyzing the stimulation distance and the sensing distance. Examples of various relationships between the stimulation distance and the sensing distance are illustrated in FIGS. 10A-10C. In various embodiments, the analysis of the stimulation distance and the sensing distance uses characteristics of the neural responses with different combinations of the stimulation distance and the sensing distance, such as illustrated in FIGS. 11A-11C. For example, stimulation control circuit 1314 can perform the electrode distance test for determining the stimulation distance relative to the sensing distance for the pair of stimulation electrode 1362 and sensing electrode 1363. Stimulation control circuit 1314 can repeat the electrode distance test for another pair of stimulation and sensing electrodes selected from electrodes 1306 until all the interested stimulation and sensing distances are determined. Stimulation control circuit 1314 can adjust control of the delivery of the neurostimulation from stimulation output circuit 1312 using a result of each electrode distance test. The adjustment can include directly adjusting values of stimulation parameters controlling the delivery of the neurostimulation and/or adjusting an algorithm that controls the stimulation values of stimulation parameters (e.g., a feedback control algorithm receiving the sensed neural signals as input).

FIG. 14 illustrates an embodiment of a stimulation control circuit 1414, which can represent an example of stimulation control circuit 1314. Stimulation control circuit 1414 can include a test controller 1464, a test processor 1466, and optionally a control adjuster 1468. Test controller 1464 can control performance of the electrode distance test including (1) sensing a first neural signal of the neural signals using sensing electrode 1363 while delivering the neurostimulation pulses using stimulation electrode 1362, and (2) sensing a second neural signal of the neural signals using stimulation electrode 1362 while delivering the neurostimulation pulses using sensing electrode 1363. Alternatively, test controller 1464 can control performance of the electrode distance test including (1) sensing a first neural signal of the neural signals using sensing electrode 1363 while delivering the neurostimulation pulses using stimulation electrode 1362, and (2) sensing a second neural signal of the neural signals using an electrode near stimulation electrode 1362 while delivering the neurostimulation pulses using an electrode near sensing electrode 1363. The electrode near stimulation electrode 1362 and stimulation electrode 1362 can be electrodes on the same lead and adjacent each other, such as without intervening electrode(s), and the electrode near sensing electrode 1363 and sensing electrode 1363 can be electrodes on the same lead and adjacent each other, such as without intervening electrode(s). Test processor 1466 can analyze the stimulation distance and the sensing distance using the first neural signal and the second neural signal. Control adjuster 1468 can adjust the control of the delivery of the neurostimulation pulses using an outcome of the analysis, thereby allowing system 1360 to automatically adjust the delivery of neurostimulation pulses using the outcome of the analysis.

To control the delivery of the neurostimulation pulses and the sensing of the neural signal for performing the electrode distance test, test controller 1464 can:
cause stimulation output circuit 1312 to deliver first neurostimulation pulses to the neural target through stimulation electrode 1362;
cause sensing circuit 1342 to sense a first neural signal from the neural target through sensing electrode 1363 while the first neurostimulation pulses are delivered through stimulation electrode 1362;
cause stimulation output circuit 1312 to deliver second neurostimulation pulses to the neural target through sensing electrode 1363 (or alternatively, through the electrode near sensing electrode 1363); and
cause sensing circuit 1342 to sense a second neural signal from the neural target through stimulation electrode 1362 (or alternatively, through the electrode near stimulation electrode 1362) while the second neurostimulation pulses are delivered through sensing electrode 1363 (or alternatively, through the electrode near sensing electrode 1363).
   The first neural signal includes first neural responses each being a response to the delivery of a pulse of the first neurostimulation pulses through stimulation electrode 1362. The second neural signal includes second neural responses each being a response to the delivery of the second neurostimulation pulses through sensing electrode 1363. Test controller 1464 can control the delivery of the first neurostimulation pulses using a first test set of stimulation parameters and control the delivery of the second neurostimulation pulses using a second test set of stimulation parameters. The second test set of stimulation parameters can be identical to the first test set of stimulation parameters except for the swap of the stimulation and sensing electrodes. The first and second sets of stimulation parameters can be determined to minimize patient perception of the electrode distance test, such as by using low pulse frequency, pulse amplitude, and/or pulse width that are sufficient for acquiring data needed for the electrode distance test.

To analyze the stimulation distance and the sensing distance, test processor 1466 can:
determine a first magnitude being a magnitude of the neural responses in the first neural signal and a second magnitude being a magnitude of the neural responses in the second neural signal; and
analyze the stimulation distance and the sensing distance using the first magnitude and the second magnitude.

In various embodiments, the magnitude of the neural responses can be measured by the amplitude, shape, and/or other related features of the neural responses, such as the neural response parameters generated by detecting and measuring ECAP features as discussed above. The analysis can include:
(1) determining whether the stimulation distance and the sensing distance are approximately equal; and, if not equal:
(2) determining a degree of difference between these the stimulation distance and the sensing distance; and/or
(3) determining which of the stimulation and sensing electrodes is closer to the neural target.
   When the first magnitude and the second magnitude are substantially equal (e.g., when the difference between the first magnitude and the second magnitude is below a specified margin), the stimulation distance and the sensing distance are approximately equal. When the first magnitude and the second magnitude are substantially different (e.g., when the difference between the first magnitude and the second magnitude is equal to or greater than the specified margin), the degree of difference (e.g., a mathematical difference, a ratio, or another expression of comparison) between these the stimulation distance and the sensing distance can be measured by the difference of the first magnitude and the second magnitude or a function of that difference (e.g., as empirically determined). The function of that difference can also allow for determination of which of the stimulation and sensing electrodes is closer to the neural target. An example for determining such a function is to generate relationships between magnitude of a neural signal and stimulation and sensing distances such as shown in FIGS. 11A-11C using simulations with a computational model. In another example, a relationship model can be developed based on empirical measurements and then used in the distance evaluations of other subjects.

The analysis can optionally include making estimates based on absolute magnitude. For example, if the degree of difference is assessed using a relative measure, the absolute magnitudes of the sensed signals in the (at least) two evoked response signals can be used to estimate the stimulation distance and the sensing distance. Additionally or alternatively, the absolute magnitudes can be used to estimate the change in the stimulation distance and the sensing distance from results of a previous electrode distance test.

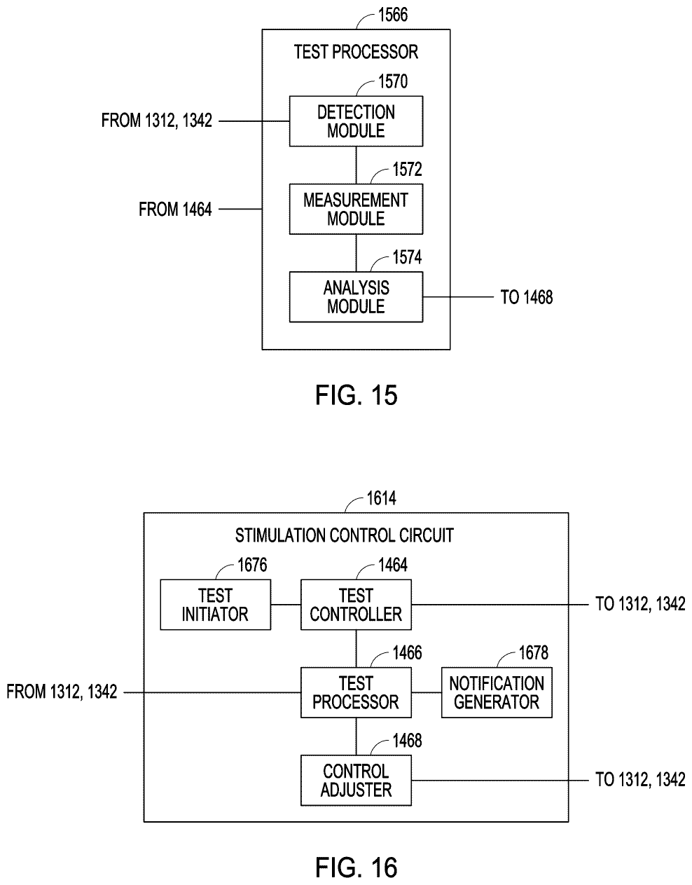

FIG. 15 illustrates an embodiment of a test processor 1566, which can represent an example of test processor 1466. Test processor 1566 can include a detection module 1570, a measurement module 1572, and an analysis module 1574.

Detection module 1570 can detect the neural responses including morphological features of the neural responses from each of the first neural signal and the second neural signal. When the neural signals include ECAPs, detection module 1570 can detect ECAPs including ECAP features each being a morphological feature of the ECAPs. Examples of the ECAP features include N1 and P2, as discussed above.

Measurement module 1572 can determine a first neural response parameter using the morphological features detected from the first neural and a second neural response parameter using the morphological features detected from the second neural signal. When the neural signals include ECAPs, detection module 1570 can determine the first neural response parameter using the ECAP features detected from the first neural signal and the first neural response parameter using the ECAP features detected from the first neural signal. Examples of the first and second neural response parameters include N1 amplitude, P1 amplitude, N1-P2 range, N1-P2 latency, N1 latency, P2 latency, CL, and AUC, as discussed above. In various embodiments, measurement module 1572 can measure each of the first and second neural response parameters directly from one or more of the ECAP features detected from the respective neural signal, calculate each of the first and second neural response parameters as a function of one or more values directly measured from the detected one or more ECAP features, or determine each of the first and second neural response parameters as a combination (e.g., a function, including a weighted function) of multiple neural response parameters directly measured from the detected one or more ECAP features.

Analysis module 1574 can produce results of the electrode distance test related to the stimulation distance and the sensing distance based on the first neural response parameter and the second neural response parameter, each of which can represent a magnitude of the neural responses. Analysis module 1574 can declare that the stimulation distance and the sensing distance are approximately equal when a difference between the first neural response parameter and the second neural response parameter is below a specified margin. Analysis module 1574 can determine a degree (or magnitude) of difference between the stimulation distance and the sensing distance by using a relationship between the degree and the difference between the first neural response parameter and the second neural response parameter. Additionally or alternatively, analysis module 1574 can determine which of the stimulation electrode and the sensing electrode is closer to the neural target (i.e., which of the stimulation distance and the sensing distance is shorter) using such a relationship. The relationship can be determined empirically, for example by modeling a system of tissue including the neural target and electrodes including the stimulation electrode and the sensing electrode, with responses of the neural target to a stimulus (e.g., a pulse) sensitive to the stimulation distance and magnitude of the sensed neural signal sensitive to the sensing distance (e.g., a computational ECAP model of the spinal cord). Simulations with this system model can then be used to determine the relationship (e.g., an estimated relationship mapping the difference between the first neural response parameter and the second neural response parameter to the difference between the stimulation distance and the sensing distance). Simulations with this system model can also be used to estimate the value of the stimulation distance and the value of the sensing distance. FIGS. 11A-11C are examples of resulting of such simulations.

Referring back to FIG. 14, control adjuster 1468 can adjust control of the delivery of the neurostimulation for the patient using one or more results of the electrode distance test provided by test processor 1466. In various embodiments, control adjuster 1468 can adjust one or more stimulation parameters of the set of stimulation parameters controlling the delivery of the neurostimulation using an outcome of the analysis of the stimulation distance and the sensing distance. For example, control adjuster 1468 can adjust an intensity of the neurostimulation based on a change in the stimulation distance relative to the sensing distance. In various embodiments, control adjuster 1468 can also adjust parameters of a control algorithm, such as a feedback control algorithm that dynamically adjusts one or more parameters of the set of the stimulation parameters based on the sensed neural signals as input. For example, for a feedback control algorithm, control adjuster 1468 can adjust a setpoint (e.g., for adjusting the intensity of the neurostimulation for maintaining a neural response parameter within a specified range) or a threshold (e.g., a value of a neural response parameter above which the intensity of the neurostimulation is to be adjusted).

FIG. 16 illustrates a stimulation control circuit 1614, which can represent another example of stimulation control circuit 1314 and a further embodiment of stimulation control circuit 1414. In addition to test controller 1464, test processor 1466, and control adjuster 1468, stimulation control circuit 1614 includes a test initiator 1676 and a notification generator 1678.

Test initiator 1676 can initiate the electrode distance test. In one embodiment, test initiator 1676 can be enabled and disabled by the user (e.g., using external programming device 802). This allows the function of performing the electrode distance test to be turned on or off as the user decides. Test initiator 1676 can initiate the electrode distance test while being enabled. In various embodiments, test initiator 1676 can, for example:
detect a change in the magnitude of the sensed neural signals and initiate the electrode distance test in response to the change exceeding a specified threshold;
detect a change in the magnitude of the accelerometer signal and initiate the electrode distance test in response to the change exceeding a specified threshold;
monitor adjustment of one or more stimulation parameters (e.g., in response to patient requests using RC 632) and initiate the electrode distance test in response to each adjustment; and/or
receive a user command (e.g., using CP 630) and initiate the electrode distance test in response to the user command.

Notification generator 1678 can generate a notification using one or more results of the electrode distance test provided by test processor 1466. In various embodiments, notification generator 1678 can generate one or more notifications related to movement of lead (including electrodes 1306) and/or changes in stimulation parameters using an outcome of the analysis of the stimulation distance and the sensing distance. For example, notification generator 1678 can generate an alarm signal in response to the outcome of the analysis indicating a condition affecting efficacy and/or safety of the neurostimulation (e.g., the difference between the stimulation distance and the sensing distance exceeding a maximum threshold indicating a displacement of a lead beyond an acceptable range). In various embodiments, each notification can be transmitted from an implantable stimulator (e.g., implantable stimulator 704) to an external programming device (e.g., external programming device 802) or other external device communicatively coupled to the implantable stimulator to be presented to the user. In various embodiments, system 1360 can automatically make adjustments in response to the notification, and/or the user need to take manual action, depending on the issue being notified.

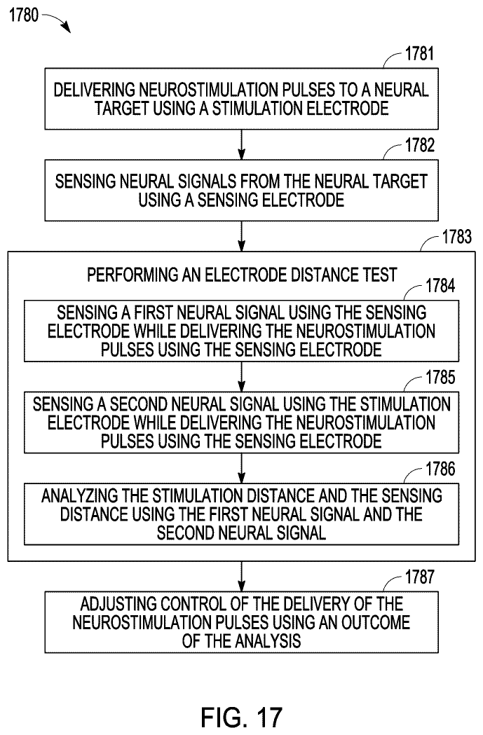

FIG. 17 illustrates an embodiment of a method 1780 for delivering neurostimulation to a neural target in a patient, including performing an electrode distance test and controlling the delivery of the neurostimulation using a result of the electrode distance test. System 1360 can be configured for performing method 1780. A non-transitory computer-readable storage medium can include instructions, which when executed by a system, such as system 1360, cause the system to perform method 1780. In one example, the instructions are included in implant storage device 746, to be executed by implantable stimulator 704 using a processor of stimulation control circuit 714.

At 1781, neurostimulation pulses are delivered to the neural target using a stimulation electrode of a plurality of electrodes (e.g., electrodes of a lead). The stimulation electrode is positioned at a stimulation distance from the neural target. At 1782, neural signals are sensed from the neural target using a sensing electrode of the plurality of electrodes. The sensing electrode is positioned at a sensing distance from the neural target. The neural signals include neural responses each evoked by the delivery of a pulse of the neurostimulation pulses.

At 1783, the electrode distance test is performed for the pair of stimulation and sensing electrodes. In various embodiments, each electrode of the plurality of electrodes can be assigned to be the stimulation electrode or the sensing electrode, and the electrode distance test can be performed for each pair of stimulation and sensing electrodes selected from the plurality of electrode. In various embodiments, the performance of the electrode distance test can be performed in response to at least one of (i) a user command for performing the electrode distance test, (ii) detection of a change in the sensed neural signals exceeding a threshold, (iii) detection of a change in an accelerometer signal (e.g., indicative of movement or posture change of the patient) exceeding a movement threshold, the accelerometer; or (iv) an adjustment of one or more stimulation parameter being made by the patient. The performance of the electrode distance test can include:
sensing a first neural signal of the neural signals using the sensing electrode while delivering the neurostimulation pulses using the stimulation electrode at 1784;
sensing a second neural signal of the neural signals using the stimulation electrode while delivering the neurostimulation pulses using the sensing electrode at 1785; and
analyzing the stimulation distance and the sensing distance using the first neural signal and the second neural signal at 1786.
   Alternatively, the performance of the electrode distance test can include:
sensing a first neural signal of the neural signals using the sensing electrode while delivering the neurostimulation pulses using the stimulation electrode at 1784;
sensing a second neural signal of the neural signals using an electrode near the stimulation electrode while delivering the neurostimulation pulses using an electrode near the sensing electrode at 1785 (the electrode near the stimulation electrode and the stimulation electrode can be electrodes on the same lead and adjacent each other, such as without intervening electrode(s), and the electrode near the sensing electrode and the sensing electrode can be electrodes on the same lead and adjacent each other, such as without intervening electrode(s)); and
analyzing the stimulation distance and the sensing distance using the first neural signal and the second neural signal at 1786.
   The analysis of the stimulation distance and the sensing distance, at 1786, can include determining a first magnitude being a magnitude of the neural responses in the first neural signal, determining a second magnitude being a magnitude of the neural responses in the second neural signal, and analyzing the stimulation distance and the sensing distance using the first magnitude and the second magnitude. The first magnitude and the second magnitude can each be determined by detecting the neural responses including morphological features of the neural responses from the respective neural signal and determine a respective neural response parameter using the morphological features detected from the respective neural signal. In one embodiment, the sensed neural signals include (ECAPs each evoked by the delivery of a pulse of the neurostimulation pulses. The morphological features include ECAP features. The first neural response parameter and the second neural response parameter are each determined using the ECAP features detected from the respective neural signal. In various embodiments, the analysis of the stimulation distance and the sensing distance includes determining whether the stimulation distance and the sensing distance are approximately equal. If the stimulation distance and the sensing distance are not approximately equal, the analysis can further include determining a degree of difference between the stimulation distance and the sensing distance and/or determining which of the stimulation and sensing electrodes is closer to the neural target.

At 1787, control of the delivery of the neurostimulation pulses can be adjusted using an outcome of the analysis performed at 1786. The adjustment can include adjusting one or more stimulation parameters directly and/or adjusting a control algorithm for adjusting one or more stimulation parameters. In various embodiments, a notification can be produced using an outcome of the analysis performed at 1786. The notification can inform the user of an issue indicated by a change in the stimulation distance and/or the sensing distance that can affect efficacy and/or safety of the neurostimulation being delivered to the patient.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation to a neural target in a patient using a plurality of electrodes, the system comprising:
a stimulation output circuit configured to deliver neurostimulation pulses to the neural target using a stimulation electrode selected from the plurality of electrodes, the stimulation electrode positioned at a stimulation distance from the neural target;
a sensing circuit configured to sense neural signals from the neural target using a sensing electrode selected from the plurality of electrodes, the sensing electrode positioned at a sensing distance from the neural target, the neural signals including neural responses each evoked by the delivery of a pulse of the neurostimulation pulses; and
a stimulation control circuit configured to control the delivery of the neurostimulation pulses, the stimulation control circuit including:
a test controller configured to control performance of an electrode distance test including sensing a first neural signal of the neural signals using the sensing electrode while delivering the neurostimulation pulses using the stimulation electrode and sensing a second neural signal of the neural signals using the stimulation electrode while delivering the neurostimulation pulses using the sensing electrode; and
a test processor configured to analyze the stimulation distance and the sensing distance using the first neural signal and the second neural signal.

2. The system of claim 1, wherein the stimulation control circuit comprises a control adjuster configured to adjust the control of the delivery of the neurostimulation pulses using an outcome of the analysis.

3. The system of claim 2, further comprising an implantable neurostimulator including the stimulation output circuit, the sensing circuit, and the stimulation control circuit.

4. The system of claim 1, wherein the test controller is configured to control the delivery of the neurostimulation pulses during the electrode distance test using stimulation parameters determined to minimize perception of stimulation by the patient.

5. The system of claim 1, wherein the test processor is configured to determine a first magnitude being a magnitude of the neural responses in the first neural signal, to determine a second magnitude being a magnitude of the neural responses in the second neural signal, and to analyze the stimulation distance and the sensing distance using the first magnitude and the second magnitude.

6. The system of claim 5, wherein the test processor is configured to determine, as an outcome of the analysis:
whether the stimulation distance and the sensing distance are approximately equal, and, if the stimulation distance and the sensing distance are not approximately equal, at least one of:
a degree of difference between the stimulation distance and the sensing distance; or
which of the stimulation and sensing electrodes is closer to the neural target.

7. The system of claim 5, wherein the test processor comprises:
a detection module configured to detect the neural responses including morphological features of the neural responses from each of the first neural signal and the second neural signal;
a measurement module configured to determine a first neural response parameter using the morphological features detected from the first neural and a second neural response parameter using the morphological features detected from the second neural signal; and
an analysis module configured to analyze the stimulation distance and the sensing distance using the first neural response parameter and the second neural response parameter.

8. The system of claim 7, wherein the neural signals include evoked compound action potentials (ECAPs) each evoked by the delivery of a pulse of the neurostimulation pulses, the detection module is configured to detect ECAPs including ECAP features each being a morphological feature of the ECAPs from each of the first neural signal and the second neural signal, and the measurement module is configured to determine the first neural response parameter using the ECAP features detected from the first neural signal and the second neural response parameter using the ECAP features detected from the second neural signal.

9. The system of claim 1, wherein the stimulation control circuit further comprises a test initiator configured to initiate the electrode distance test in response to at least one of:
an initiation command;
a change in the sensed neural signal exceeding a specified threshold;
a change in an accelerometer signal indicative of a movement or posture of the patient exceeding a specified threshold; or
one of the one or more parameters of the set of stimulation parameters being adjusted by the patient.

10. The system of claim 1, wherein the stimulation control circuit further comprises a notification generator to produce a notification using an outcome of the analysis.

11. A method for delivering neurostimulation to a neural target in a patient, the method comprising:
delivering neurostimulation pulses to the neural target using a stimulation electrode of a plurality of electrodes, the stimulation electrode positioned at a stimulation distance from the neural target;
sensing neural signals from the neural target using a sensing electrode of the plurality of electrodes, the sensing electrode positioned at a sensing distance from the neural target, the neural signals including neural responses each evoked by the delivery of a pulse of the neurostimulation pulses;
performing an electrode distance test including sensing a first neural signal of the neural signals using the sensing electrode while delivering the neurostimulation pulses using the stimulation electrode and sensing a second neural signal of the neural signals using the stimulation electrode while delivering the neurostimulation pulses using the sensing electrode; and
analyzing the stimulation distance and the sensing distance using the first neural signal and the second neural signal.

12. The method of claim 11, further comprising adjusting control of the delivery of the neurostimulation pulses using an outcome of the analysis.

13. The method of claim 11, wherein analyzing the stimulation distance and the sensing distance using the first neural signal and the second neural signal comprises:
determining a first magnitude being a magnitude of the neural responses in the first neural signal;
determining a second magnitude being a magnitude of the neural responses in the second neural signal; and
analyzing the stimulation distance and the sensing distance using the first magnitude and the second magnitude.

14. The method of claim 13, wherein analyzing the stimulation distance and the sensing distance comprises determining whether the stimulation distance and the sensing distance are approximately equal.

15. The method of claim 14, wherein analyzing the stimulation distance and the sensing distance comprises determining, if the stimulation distance and the sensing distance are not approximately equal, at least one of:
a degree of difference between the stimulation distance and the sensing distance; or
which of the stimulation and sensing electrodes is closer to the neural target.

16. The method of claim 13, wherein determining the first magnitude and determining the second magnitude comprise:
detecting the neural responses including morphological features of the neural responses from each of the first neural signal and the second neural signal;
determining a first neural response parameter as the first magnitude using the morphological features detected from the first neural signal; and
determining a second neural response parameter as the second magnitude using the morphological features detected from the second neural signal.

17. The method of claim 16, wherein sensing the neural signals comprises sensing neural signals including evoked compound action potentials (ECAPs) each evoked by the delivery of a pulse of the neurostimulation pulses, detecting the neural responses comprises detecting ECAP features each being a morphological feature of the ECAPs, determining the first neural response parameter comprises determining the first neural response parameter using the ECAP features detected from the first neural signal; and determining the second neural response parameter comprises determining the second neural response parameter using the ECAP features detected from the second neural signal.

18. The method of claim 11, further comprising initiating the performance of the electrode distance test in response to at least one of:
a user command for performing the electrode distance test;
detection of a change in the sensed neural signals exceeding a sensing threshold;
detection of a change in an accelerometer signal exceeding a movement threshold, the accelerometer signal indicative of movement or posture of the patient; or
an adjustment of one or more specified parameters of the set of stimulation parameters being made by the patient.

19. The method of claim 11, further comprising producing a notification using an outcome of the analysis.

20. A non-transitory computer-readable storage medium including instructions, which when executed by a system, cause the system to perform a method for delivering neurostimulation to a neural target in a patient, the method comprising:
delivering neurostimulation pulses to the neural target using a stimulation electrode of a plurality of electrodes, the stimulation electrode positioned at a stimulation distance from the neural target;
sensing neural signals from the neural target using a sensing electrode of the plurality of electrodes, the sensing electrode positioned at a sensing distance from the neural target, the neural signals including neural responses each evoked by the delivery of a pulse of the neurostimulation pulses;
performing an electrode distance test including sensing a first neural signal of the neural signals using the sensing electrode while delivering the neurostimulation pulses using the stimulation electrode and sensing a second neural signal of the neural signals using the stimulation electrode while delivering the neurostimulation pulses using the sensing electrode; and
analyzing the stimulation distance and the sensing distance using the first neural signal and the second neural signal.
